(19) United States
(12) Patent Application Publication
(10) Pub. No.: US 2013/0331704 A1
(43) Pub. Date: Dec. 12, 2013

(54) FLEXIBLE ULTRASOUND TRANSDUCER DEVICE

(71) Applicant: Aram T. Salzman, Belmont, MA (US)

(72) Inventor: Aram T. Salzman, Belmont, MA (US)

(21) Appl. No.: 13/991,794

(22) PCT Filed: Dec. 6, 2011
(86) PCT No.: PCT/US11/63540

§ 371 Date: Aug. 19, 2013

Related U.S. Application Data
(60) Provisional application No. 61/420,062, filed on Dec. 6, 2010.

(51) Int. Cl. A61B 8/00; A61B 8/02; A61B 8/08

(52) U.S. Cl. CPC A61B 8/4494; A61B 8/4281; A61B 8/4236; A61B 8/5223; A61B 8/0866; A61B 8/02; A61B 8/4477; A61B 8/54; A61B 8/565
USPC 600/459

(57) ABSTRACT

Exemplary embodiments provide a flexible, ambulatory and non-invasive ultrasound transducer device attachable to a user's body for transmitting ultrasound waves and receiving echoed ultrasound waves for performing non-invasive monitoring or imaging of the internal structures of the user's body. One or more ultrasound transducers are provided on a flexible, soft and thin backing layer that is adhered conformably and closely to a user's body so that the transducers are maintained in direct and intimate contact with the user's body. An exemplary device is used to monitor the heartbeat of a fetus inside a pregnant woman's womb.

### RELATED APPLICATIONS

This application is a non-provisional of and claims priority to U.S. Provisional Application Ser. No. 61/420,062, filed Dec. 6, 2010, titled “Flexible Ultrasound Transducer Array for Imaging and Fetal Monitoring,” the entire contents of which are expressly incorporated herein in their entirety by reference.

### BACKGROUND

The term “ultrasound” typically applies to sound waves with frequencies above the audible range of human hearing, which is about 20 kHz. Many medical monitoring, imaging and diagnostic techniques rely on ultrasound waves to detect and visualize subcutaneous structures in the body including, but not limited to, fetuses, muscles, tendons, internal organs, and the like. Ultrasound in the frequency range of about 2 MHz to about 20 MHz is typically used in imaging internal human organs.

A medical device employing ultrasound typically generates ultrasound waves using ultrasound transducers that convert electrical power into acoustical output power. The ultrasound waves are transmitted into a user's body and are reflected back to the medical device from internal structures in the user's body. The reflected ultrasound waves are typically converted into instructions encoded in electrical signals which are processed to image and/or monitor subcutaneous structures in the user's body.

### SUMMARY

In accordance with an exemplary embodiment, a flexible ultrasound transducer device is provided. In an exemplary embodiment, the exemplary flexible ultrasound transducer device uses ultrasound Doppler technology to detect and monitor the heartbeat of a fetus inside a pregnant woman's womb. The exemplary device may monitor the fetal heartbeat at a particular time or over a period of time. The exemplary device may reliably monitor the fetal heartbeat across an array of ultrasound transducers even as the position of the fetus changes within the womb and/or as the mother's position changes.

The flexible ultrasound transducer device includes a flexible backing layer having an adhesive proximal surface configured for direct attachment to a user's body and conformable to the user's body. In an exemplary embodiment, the flexible ultrasound transducer device is reusable between sessions of ultrasound monitoring. In an exemplary embodiment, the backing layer is conformable and attachable to any location of the user's body in any desired orientation.

The flexible ultrasound transducer device also includes one or more ultrasound transducers affixed to the proximal surface of the flexible backing layer and configured so that, upon attachment of the flexible backing layer to the user's body, the ultrasound transducers are maintained in direct and intimate contact with the user's body. The transducers may be microelectromechanical systems (MEMS) ultrasound transducers or piezoelectric ultrasound transducers in some exemplary embodiments.

In some exemplary embodiments, the backing layer may support a single ultrasound transducer. In other exemplary embodiments, the backing layer may support a plurality of ultrasound transducers ranging, in some embodiments, from two to a million transducers. Some exemplary numbers of transducers include, but are not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, and the like. The use of an array of multiple transducers in some exemplary embodiments allows broad coverage over the user's body for monitoring and/or imaging. The broad coverage provided by exemplary devices avoids the need for re-positioning the transducers or the device over time, and allows easy and efficient imaging and monitoring.

In an exemplary embodiment, the ultrasound transducers are configured in a rectangular grid arrangement on the backing layer to cover a desired portion of the user's body. In another exemplary embodiment, the ultrasound transducers are configured in a linear strip.

The flexible ultrasound transducer device includes an ultrasound gel provided on the proximal surface of the flexible backing layer and interspersed among the ultrasound transducers to facilitate transmission of the ultrasound waves. Upon attachment of the flexible backing layer to the user's body, the ultrasound gel is maintained in direct and close contact with the user's body and is prevented from leaking by close adherence of the edges of the backing layer to the user's body.

The flexible ultrasound transducer device includes an output data processing module coupled to the transducers for receiving output data from the transducers, and programmed to monitor one or more physiological aspects of a fetus in the user's body based on the output data. The one or more physiological aspects of the fetus includes one or more of the following: an ultrasound image of the fetus, a structure of the fetus, a location of the fetus, a movement of the fetus, fetal heartbeat, fetal heart rate, breathing of the fetus, lung movement of the fetus, a depth in the user's body of a pocket of amniotic fluid, tone/flexion of the fetus, a reactivity of a heart rate and/or heartbeat of the fetus to movement, and a biophysical profile of the fetus.

In an exemplary embodiment, the flexible ultrasound transducer device includes an output data processing module programmed to receive a first set of output data from a first transducer and a second set of output data from a second transducer among the plurality of transducers. The output data processing module is programmed to automatically perform a quality assessment of the first and second sets of output data, automatically determine that the first set of output data has a higher quality and/or suitable for data processing than the second set of output data, and automatically determine that the first transducer is more suitable for ultrasound data processing than the second transducer. The output data processing module may be programmed to use output data received from the first transducer to perform the ultrasound data processing, and to automatically exclude output data received from the second transducer from the ultrasound data processing. The output data processing module may be programmed to automatically instruct a control module to deactivate the second transducer based on the comparative quality assessment between the first and second transducers.

In an exemplary embodiment, ultrasound data processing performed by the output data processing module includes fetal monitoring and/or fetal imaging.

In an exemplary embodiment, the flexible ultrasound transducer device includes a control module coupled to the transducers and programmed to allow selective deactivation of a subset of the plurality of transducers.

In accordance with another exemplary embodiment, a method is provided for performing ultrasound monitoring and/or imaging of a user's body. In an exemplary embodiment, the exemplary method uses ultrasound Doppler technology to detect and monitor the heartbeat of a fetus inside a pregnant woman's womb. The exemplary method may monitor the fetal heartbeat at a particular time or over a period of time. The exemplary method may reliably monitor the fetal heartbeat across an array of ultrasound transducers even as the position of the fetus changes within the womb and/or as the mother's position changes.

The method includes providing a flexible ultrasound transducer device that includes a flexible backing layer having an adhesive proximal surface configured for direct attachment to the user's body and conformable to the user's body. The flexible ultrasound transducer device also includes a set of one or more ultrasound transducers affixed to the proximal surface of the flexible backing layer. The method also includes attaching the adhesive proximal surface of the flexible backing layer to the user's body in a conforming manner, so that the one or more ultrasound transducers are maintained in direct and intimate contact with the user's body. The method further includes activating at least one of the one or more transducers in order to perform ultrasound monitoring and/or imaging of the user's body.

In an exemplary embodiment, the method includes providing an ultrasound gel on the proximal surface of the flexible backing layer and interspersed among the one or more ultrasound transducers. Upon attachment of the flexible backing layer to the user's body, the ultrasound gel is maintained in direct and intimate contact with the user's body and is prevented from leaking by adherence of edges of the backing layer to the user's body.

In an exemplary embodiment, the method includes receiving, at an output data processing module coupled to the one or more transducers, output data generated by the one or more transducers and monitoring one or more physiological aspects of a fetus in the user's body based on the output data.

In an exemplary embodiment, the method includes receiving a first set of output data from a first transducer within the set of transducers, and receiving a second set of output data from a second transducer within the set of transducers. The method also includes automatically performing a quality assessment of the first and second sets of output data, and automatically determining that the first set of output data has a higher quality and/or suitability for use in ultrasound data processing than the second set of output data. The method includes automatically determining that the first transducer is more suitable for the ultrasound data processing than the second transducer. In an exemplary embodiment, the method includes using output data received from the first transducer to perform the ultrasound data processing, and automatically excluding output data received from the second transducer from the ultrasound data processing. In an exemplary embodiment, the method includes automatically instructing a control module to deactivate the second transducer.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings, in which:

FIG. 1 illustrates a schematic side view of an exemplary microelectromechanical systems (MEMS) ultrasound transducer.

FIG. 2A illustrates a schematic side view of an exemplary flexible ultrasound transducer device in a packaged state.

FIG. 2B illustrates a schematic side of the exemplary device of FIG. 2A in use, in which an adhesive proximal surface of a backing layer is attached to a user contact region on a user's body.

FIG. 3A illustrates a top view of an exemplary flexible ultrasound transducer device, showing a surface of a backing layer distal to a user contact region.

FIG. 3B illustrates a bottom view of the exemplary flexible ultrasound transducer device of FIG. 3A, showing a surface of the backing layer proximal to a user contact region.

FIG. 4A illustrates a schematic front view of a pregnant woman's abdomen with an exemplary flexible ultrasound transducer device attached to the abdomen for fetal monitoring.

FIG. 4B illustrates a schematic side view of a pregnant woman's abdomen with the exemplary ultrasound transducer device of FIG. 4A attached to the abdomen for fetal monitoring.

FIG. 5 is a flowchart illustrating an exemplary method for assembling an exemplary ultrasound transducer device.

FIG. 6 is a flowchart illustrating an exemplary method for using an exemplary to ultrasound transducer device.

FIG. 7 illustrates a schematic of a single exemplary ultrasound transducer provided in an exemplary flexible ultrasound transducer device.

FIG. 8 illustrates a schematic of an exemplary linear strip of ultrasound transducers provided in an exemplary flexible ultrasound transducer device.

FIG. 9 is a schematic front view of a pregnant woman's abdomen on which two exemplary linear strips of ultrasound transducers are placed spaced from each other.

FIG. 10 illustrates a schematic of an exemplary two dimensional grid array of ultrasound transducers provided in an exemplary flexible ultrasound transducer device.

FIG. 11 is a block diagram representing an exemplary computing device that may be used to perform any of the methods provided by exemplary embodiments.

FIG. 12 is a block diagram representing an exemplary network environment suitable for a distributed implementation of exemplary embodiments.

### DETAILED DESCRIPTION

Exemplary embodiments provide a flexible, ambulatory and non-invasive ultrasound transducer device that may be used to transmit ultrasound waves to a user's body and receive echoed ultrasound waves for performing non-invasive monitoring and/or imaging of the internal structures of the user's body. In an exemplary embodiment, the exemplary flexible ultrasound transducer device uses ultrasound Doppler technology to detect and monitor the heartbeat of a fetus inside a pregnant woman's womb. The exemplary device may monitor the fetal heartbeat at a particular time or over a period of time. The exemplary device may reliably monitor the fetal heartbeat across an array of ultrasound transducers even as the position of the fetus changes within the womb and/or as the mother's position changes.

In an exemplary flexible ultrasound transducer device, one or more ultrasound transducers may be provided on a flexible, soft and thin backing layer that may be adhered directly and intimately to a user's body so that the backing layer closely conforms to the contours of the body. The flexible and thin backing layer allows the transducers to be maintained in direct and close contact with the user's body. “Direct” contact refers to contact of the transducers with the user's body without any intervening component of the device (e.g., the backing layer, a garment, etc.) being disposed between the transducers and the body. Any suitable ultrasound transducers may be used in exemplary embodiments including, but not limited to, microelectromechanical systems (MEMS) ultrasound transducers, piezoelectric ultrasound transducers, and the like.

Some conventional ultrasound devices use Doppler technology in fetal monitor probes to measure fetal heart beat in the mother's womb. Some conventional discs are about 2 cm thick and about 5 cm to about 10 cm in diameter or length, and are configured as thick discs that offer limited coverage of the changing location of the fetus in the womb. Since these conventional devices provide limited coverage of the fetal location, it is necessary to re-position the conventional devices as the location of the fetus and/or the mother changes over time, making fetal imaging and monitoring time and labor-intensive for nurses, midwives and obstetricians.

Successful ultrasound imaging and monitoring requires close contact between the ultrasound transducers and the user's body for the generation of output signals suitable for monitoring and imaging purposes. Some conventional ultrasound devices provide the ultrasound sensors or transducers in a separate garment, such as a belt, that is worn over the user's body. However, providing the ultrasound transducers in a separate garment in these conventional devices prevents the transducers from directly contacting the user's skin and precludes a close contact of the sensors with the skin. This results in output signals that are not usable for successful ultrasound monitoring and imaging in conventional devices.

Furthermore, since the ultrasound transducers in some conventional devices are provided in a separate garment, ultrasound gel cannot be provided between the transducers and the user's skin to facilitate transmission of the ultrasound waves. Even if an ultrasound gel were provided, a conventional device providing transducers on belts would be unable to contain the ultrasound gel within the device as the gel would tend to leak out from the edges of the belt, which would dry out the gel and render the device unsuitable for use in ultrasound imaging and/or monitoring.

Exemplary ultrasound transducer devices avoid the shortcomings of conventional devices by adhering the ultrasound transducers directly and intimately to the user's body, i.e., without the use of a separate garment worn over the body. For example, exemplary transducers may be supported on and adhered to the body using a soft, thin and flexible adhesive backing layer that may be applied to and peeled-off from the user's skin. Exemplary backing layers are maintained in close contact with the skin during use and conform to the contours of the user's body. In addition, since exemplary ultrasound transducer devices provide direct and intimate contact between the transducers and the user's skin, an ultrasound gel may be provided in the devices surrounding the transducers for facilitating transmission of the ultrasound waves.

Exemplary flexible ultrasound transducer devices may be used in many suitable medical applications. In some exemplary embodiments, exemplary transducer devices may be used to non-invasively image any subcutaneous structure in a user's body. In some exemplary embodiments, exemplary transducer devices may be placed on a pregnant user's abdomen, and may be used to non-invasively image and/or monitor physiological aspects of the fetus and/or the mother. In an exemplary embodiment, the devices may be used to detect and monitor the heartbeat of a fetus in the user's womb.

Exemplary array configurations of exemplary transducers allow the ultrasound waves to provide wide coverage of the womb, and allow monitoring and/or imaging without having to first locate the fetus within the body and without having to adjust the location of the transducers over time.

One of ordinary skill in the art will appreciate that exemplary flexible ultrasound transducer devices may be used to perform different types of fetal imaging and monitoring, and non-fetal imaging and monitoring of a user's body. Exemplary physiological aspects of a fetus that may be monitored using exemplary embodiments include, but are not limited to, detecting and monitoring the fetal heartbeat, generating one or more ultrasound images of the fetus, determining a structure of the fetus, determining a location of the fetus, detecting a movement of the fetus, determining fetal heart rate, detecting the breathing of the fetus, detecting lung movement of the fetus, determining a depth in the user's body of a pocket of amniotic fluid, determining the tone/flexion of the fetus, determining a reactivity of a heart rate and/or heartbeat of the fetus to movement, determining a biophysical profile of the fetus, and the like.

Exemplary embodiments are described below with reference to certain illustrative embodiments.

### I. Exemplary Ultrasound Transducers

Ultrasound transducers usable in exemplary flexible ultrasound transducer devices may employ any suitable technique to generate ultrasound waves and to receive reflected ultrasound waves.

In one example, exemplary ultrasound transducers may be microelectromechanical systems (MEMS) transducers. MEMS are small mechanical devices driven by electrical energy.

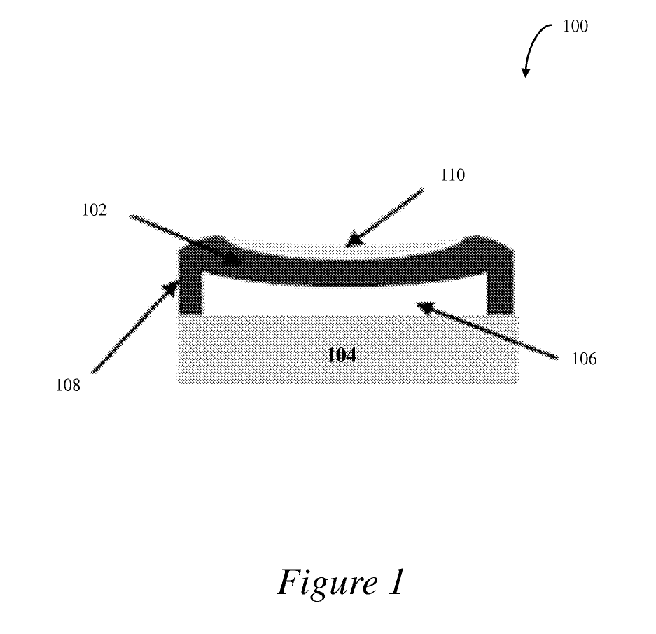

FIG. 1 illustrates a schematic side view of an exemplary MEMS ultrasound transducer 100. In an exemplary MEMS ultrasound transducer 100, a silicon ultrasonic sensor may include a small drum-like structure having a thin, ultra-sensitive nitride membrane 102. The drum structure may be provided with a silicon substrate 104 that forms a bottom electrode, a central cavity 106 surrounded by a silicon nitride support 108, and an aluminum electrode 110 suspended over the cavity 106.

The drum structure is a capacitive structure that operates under an applied electrostatic field. A bias voltage applied across the top and bottom electrodes establishes an electric field that creates a variable tension in the nitride membrane, and causes the membrane to vibrate and emit ultrasound waves. Conversely, during reception of reflected ultrasound waves, an ultrasound wave impinges on the top electrode membrane and causes movement in the membrane. The movement alters the capacitance of the sensor and creates an output electrical current. Measurement of aspects of the generated electrical current allows monitoring and imaging of the subcutaneous structures of the body that caused reflection of the ultrasound waves. Other types of MEMS transducers may also be used in exemplary ultrasound transducer devices.

Use of MEMS transducers allow minimization of the weight and mass of exemplary devices as the transducers are miniature in size. Some exemplary MEMS ultrasound transducers may range in length and width from about 0.01 mm to about 5 mm, but are not limited to this exemplary range. Some exemplary MEMS ultrasound transducers may range in thickness from about 0.01 mm to about 0.5 mm, but are not limited to this exemplary range.

Since the MEMS transducers are miniature in size, exemplary ultrasound transducer devices may be attached to the user's body directly (i.e., without the use of a separate garment) using a flexible adhesive backing layer and may be provided in direct and intimate contact with the body in use. In addition, the MEMS transducers are efficient at energy management and do not suffer from the problem of self-heating. These advantages may not be afforded by conventional ultrasound transducers that are larger, unwieldy and bulky, and that may not be possible to provide in close contact with the body or to be attached to the body using a flexible adhesive backing layer.

In another example, exemplary ultrasound transducers may be piezoelectric ultrasound transducers. An exemplary piezoelectric ultrasound transducer may include one or more plates formed of one or more piezoelectric materials such as lead zirconate titanate (PZT) ceramics, electrodes, matching layers, and backing materials. When an electrical field is applied to the electrodes on opposite sides of the piezoelectric plate, the thickness of the plate may expand or contract depending on the polarity of the field. The expansion or contraction of the plate may cause mechanical vibrations that, in turn, generate ultrasound waves. Conversely, during reception of reflected ultrasound waves, an ultrasound wave impinges on the piezoelectric plate and causes the piezoelectric material to generate an output electrical current. Measurement of aspects of the generated electrical current allows monitoring and imaging of the subcutaneous structures of the body that caused reflection of the ultrasound waves.

Some exemplary piezoelectric ultrasound transducers may range in length and width from about 5 mm to about 30 mm, but are not limited to this exemplary range. Some exemplary piezoelectric ultrasound transducers may range in thickness from about 0.1 mm to about 2 mm, but are not limited to this exemplary range.

Other types of transducers may also be used in exemplary ultrasound transducer devices.

### II. Exemplary Embodiments of Flexible Ultrasound Transducer Devices

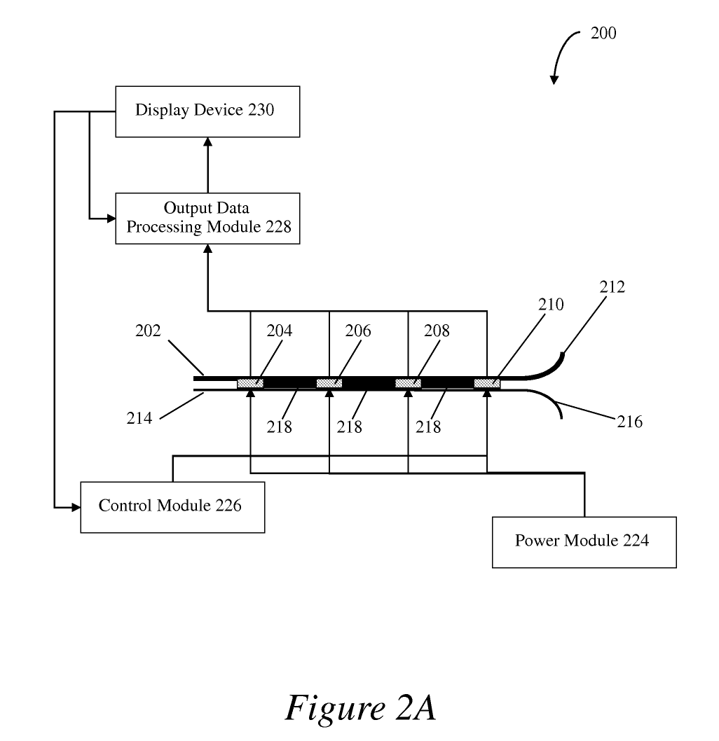

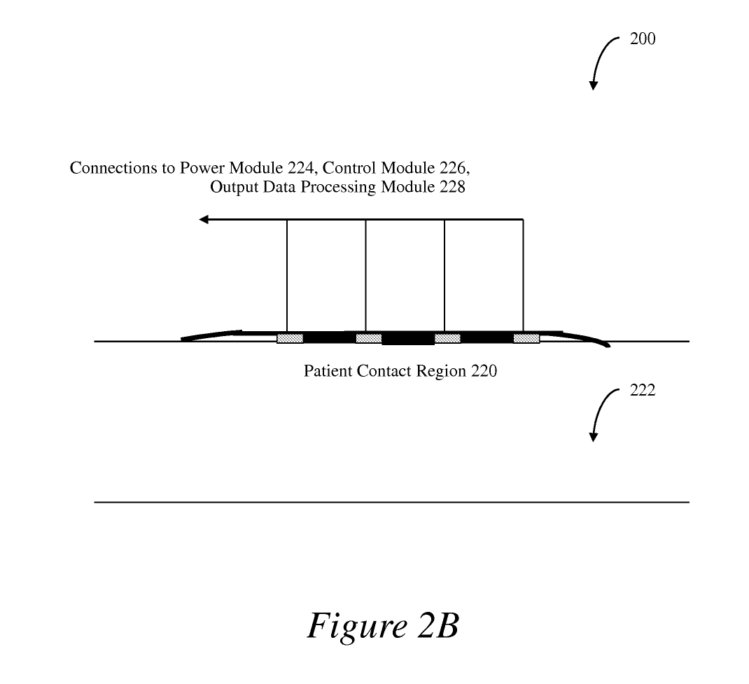

FIG. 2A illustrates a schematic side view of an exemplary flexible ultrasound transducer device 200 that may be used to generate ultrasound waves and to receive ultrasound waves reflected back from a user's body. One of ordinary skill in the art will recognize that the schematic is provided for illustrative purposes and that the different components shown in the schematic are not drawn to scale.

The ultrasound transducer device 200 may include a thin, soft, flexible backing layer 202 for supporting an arrangement of one or more ultrasound transducers 204, 206, 208, 210. In an exemplary embodiment, the backing layer 202 may take the form of a bandage with an adhesive surface that may be easily adhered to and peeled-off from the user's body.

In some exemplary embodiments, the backing layer 202 may support a single ultrasound transducer. In other exemplary embodiments, the backing layer 202 may support a plurality of ultrasound transducers ranging, in some embodiments, from two to a million transducers. Some exemplary numbers of transducers include, but are not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, and the like. The use of an array of multiple transducers in some exemplary embodiments allows broad coverage over the user's body for monitoring and/or imaging. The broad coverage provided by exemplary devices avoids the need for re-positioning the transducers or the device over time, and allows easy and efficient imaging and monitoring.

In an exemplary embodiment, a proximal surface of the backing layer 202 configured for adhering to the user contact region on the user's body may be provided with an adhesive so that the transducers may be affixed to the backing layer with the adhesive. The backing layer 202 itself may also be adhered directly to the user contact region using the adhesive.

Some exemplary backing layers 202 may having a thickness ranging from about 0.5 mm to about 3.0 mm, but exemplary thicknesses of backing layers are not limited to this exemplary range. The thin, soft and flexible structure of an exemplary backing layer allows the backing layer to be conformable and attachable to any location of the user's body in any desired orientation. In contrast to conventional devices that provide ultrasound transducers on a separate garment like a belt, the thin design of an exemplary backing layer allows direct and intimate contact of the exemplary backing layer and exemplary ultrasound transducers with the user's body.

In an exemplary embodiment, the adhesive backing layer 202 may include a non-adhesive removal mechanism 212, e.g., a tab, that is not adhesive. The non-adhesive removal mechanism 212 may be gripped by a user and pulled to remove the transducer device from the user's body.

The backing layer 202 may be formed of any suitable thin, soft and flexible material including, but not limited to, plastic, polymeric materials, elastic fabric, cloth, leather, rubber, elastic, and the like. The use of a thin flexible backing layer allows the device 200 to conform to the contours of the user's body, provide the transducers in direct and close contact with the body, and generate ultrasound waves and signals encoded in electrical instructions and data that result in accurate and precise monitoring and/or imaging of the user's body. Since the backing layer 202 is made of a thin, soft and flexible material and is not provided as a separate garment that needs to be worn by the user, the backing layer is conformable and attachable to any desired location on the user's body in any desired orientation. The backing layer 202 may also be peeled off from one location of the user's body for repositioning at a different location and/or in a different orientation.

In an exemplary embodiment, one or more ultrasound transducers and/or the backing layer may be formed of a reusable material so that the flexible ultrasound transducer device may be reused on the same user's body or on a different user's body. Exemplary reusable materials may include, but are not limited to, polyurethane, nylon, polystyrene, polyester, polypropylene, plastic resins, and the like. The device may be sterilized before reuse. In another exemplary embodiment, one or more ultrasound transducers and/or the backing layer may be formed of a disposable material so that the flexible ultrasound transducer device may be disposed of after each use. Exemplary disposable materials may include, but are not limited to, generic polyurethane, cotton, nylon, medical foams, polypropylene, rayon, polyester materials, the Tegaderm™ medical dressing manufactured by 3M, and the like.

The backing layer 202 may have any suitable size, shape and configuration for covering a desired region and area of the user's body.

In a packaged state before the transducer device is placed in use, the adhesive backing layer 202 may be covered by a sterile protective film 214 which preserves the adhesive nature of the backing layer 202. The protective film 214 may also maintain sterility of the one or more transducers and the backing layer. In an exemplary embodiment, the protective film 214 may include a removal mechanism 216 that may be gripped by a user and pulled to remove the protective film 214 from the backing layer 202. This exposes the adhesive surface of the backing layer 202 and allows the device 200 to be attached to the user contact region on the user's body.

In the packaged state, the device 200 may include conductive ultrasound gel 218 provided between the backing layer 202 and the protective film 214 to assure efficient transmission of the ultrasound waves between the ultrasound transducers and the user's body. The ultrasound gel may be interspersed among the transducers on the backing layer. Upon removal of the protective film 214, the ultrasound gel 218 is exposed and may be placed in direct contact with the user contact region on the user's body. The one or more transducers and gel may be adhered to the user contact region to directly contact the user's skin. When the device 200 is applied to the user contact region, the ultrasound gel is tightly confined between the proximal surface of the backing layer 202 and the user contact region and is interspersed among the one or more transducers. The adhesive nature of the proximal surface of the backing layer 202 and the close contact of the edges of the backing layer 202 with the user contact region allow the ultrasound gel to be maintained in contact with the user's body during use and prevents the gel from leaking out from the sides or edges of the device.

FIG. 2A illustrates the exemplary device 200 in a packaged state in which the protective film 214 is in place over the proximal surface of the backing layer 202. FIG. 2B illustrates the exemplary device 200 in use, in which the protective film 214 is removed from the proximal surface of the backing layer 202 and in which the adhesive proximal surface is attached to a user contact region 220 on a user's body 222.

The exemplary ultrasound transducer device 200 may include a power module 224 separately couplable to each ultrasound transducer for providing electrical energy for operation of the ultrasound transducers. Exemplary power modules 224 may include, but are not limited to, one or more portable batteries, electrical mains, and the like. In an exemplary embodiment, the power module 224 may be provided integrally with the one or more ultrasound transducer, for example, so that the power module 224 may be directly or indirectly placed on or in the neighborhood of the user's body. In another exemplary embodiment, the power module 224 may be provided separately from the one or more ultrasound transducers, for example, so that the power module may be located remotely from the user's body.

The exemplary ultrasound transducer device 200 may include a control module 226 separately couplable to each ultrasound transducer and programmed to provide instructions to control the operation and functioning of the transducers. The control module may control, for example, activation of the transducer, deactivation of the transducer, supplying or cutting of power supply to the transducer, frequency settings, duration settings, scan settings, and the like. In an exemplary embodiment, a common control module may be provided to control all of the transducers in the device. In another exemplary embodiment, a plurality of control modules may be provided to separately control all of the transducers or to separately control different subsets of the transducers.

In an exemplary embodiment, the control module 226 may be provided integrally with the one or more ultrasound transducers, for example, so that the control module 226 may be directly or indirectly placed on or in the neighborhood of the user's body. In another exemplary embodiment, the control module 226 may be provided separately from the one or more ultrasound transducers, for example, so that the control module may be located remotely from the user's body.

The exemplary ultrasound transducer device 200 may include an output data processing module 228 separately couplable to each ultrasound transducer and programmed to process output data received from the transducers. The output data processing module may be programmed to perform one or more data processing activities on the output data received from the transducers including, but not limited to, detecting the fetal heartbeat at a particular time or over time, generating one or more images, detecting and monitoring the structure, location and movements of a fetus at a particular time or over time, detecting fetal heart rate at a particular time or over time, detecting breathing and lung movement of a fetus at a particular time or over time, detecting the depth of pockets of amniotic fluid, detecting tone/flexion of a fetus at a particular time or over time, detecting reactivity of fetal heart rate and/or heartbeat to movement at a particular time or over time, generating a biophysical profile for a fetus based on one or more of the above information, assessing quality of output data received from a particular transducer at a particular time or over time, determining which transducer is generating suitable or unsuitable output data at a particular time or over time, and the like.

In some exemplary embodiments, the output data processing module 228 may communicate with the control module 226 over a network communication network or via other communication means. The output data processing module 228 may provide one or more instructions to the control module 226 to selectively control the operation of one or more transducers, e.g., by selectively deactivating or activating one or more of the transducers.

In an exemplary embodiment, the output data processing module 228 may be provided integrally with the one or more ultrasound transducers, for example, so that the output data processing module 228 may be directly or indirectly placed on or in the neighborhood of the user's body. In another exemplary embodiment, the output data processing module 228 may be provided separately from the one or more ultrasound transducers, for example, so that the output data processing module may be located remotely from the user's body.

The exemplary ultrasound transducer device 200 may include a display device 230 couplable to the output data processing module 228. The display device 230 may be used to display output data received from the transducers and/or processed data generated by the output data processing module 228 based on the raw output data. For example, the display device 230 may display fetal heartbeat at a particular time or over time, one or more images generated based on the output data, processed data on the structure, location and movements of a fetus at a particular time or over time, fetal heart rate at a particular time or over time, breathing and lung movement of a fetus at a particular time or over time, location and depth of pockets of amniotic fluid at a particular time or over time, tone/flexion of a fetus at a particular time or over time, reactivity of fetal heart rate and/or fetal heartbeat to movement at a particular time or over time, a biophysical profile for a fetus at a particular time or over time based on any of the above information, quality indicators for the output data received from a particular transducer, selection of one or more transducers at a particular time or over time that are currently generating improved output data, and the like.

In some exemplary embodiments, the display device 230 may display a graphical or textual user interface for allowing a user to query the output data processing module 228 for raw output data from the transducers and/or processed data. The user may use the user interface to provide instructions to the output data processing module 228 to process output data from a selected subset of the transducers. The user may also use the user interface to provide instructions to the control module 226, for example, to activate or deactivate one or more of the transducers.

In some exemplary embodiments, a flexible ultrasound transducer assembly including one or more transducers (e.g., transducers 204, 206, 208, 210) attached to a backing layer (e.g., backing layer 202) may be provided as a separate unit that may be compatible with and plugged into one or more additional components of the device. For example, the exemplary backing layer and the one or more transducers may be compatible with and plugged into the power module 224, the control module 226, and the output data processing module 228.

In some exemplary embodiments, a flexible ultrasound transducer assembly including one or more transducers (e.g., transducers 204, 206, 208, 210) attached to a backing layer (e.g., backing layer 202) may be provided as a separate unit that may be compatible with and plugged into existing ultrasound monitoring and/or imaging systems. Exemplary existing ultrasound monitoring and/or imaging systems that an exemplary backing layer and transducer assembly may be plugged into include, but are not limited to, the Corometrics™ ultrasound monitoring system from General Electric Company, the Avalon fetal monitoring system from Koninklijke Philips Electronics N.V., and the like. That is, existing ultrasound monitoring and/or imaging systems may be configured to provide exemplary backing layers fitted with one or more ultrasound transducers.

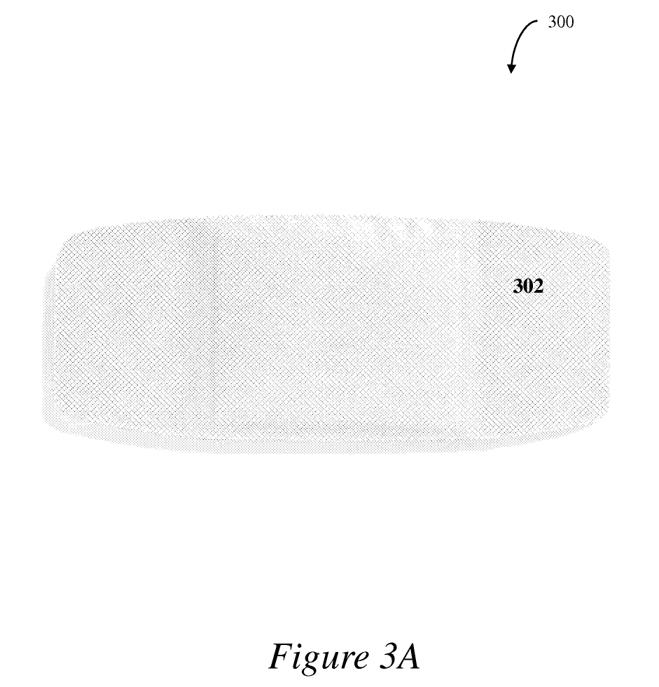

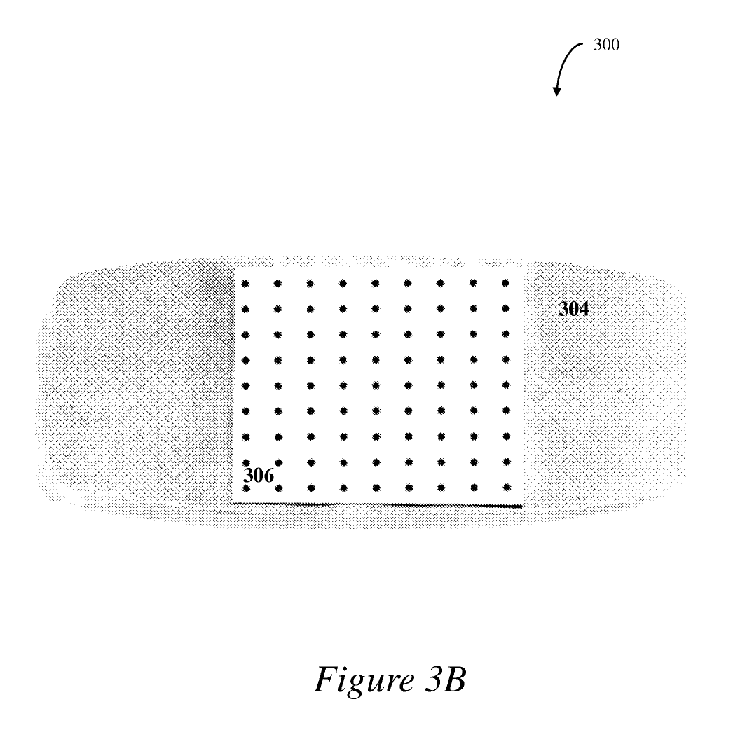

FIG. 3A illustrates a top view of an exemplary flexible ultrasound transducer device 300, showing a surface 302 of a backing layer distal to a user contact region. That is, on application of the device 300 to a user's body, the distal surface 302 may not be in contact with the user contact region and may face away from the user contact region. The distal surface 302 may be non-adhesive.

FIG. 3B illustrates a bottom view of the exemplary flexible ultrasound transducer device 300 of FIG. 3A, showing a surface 304 of the backing layer proximal to and configured for adhering to a user contact region on a user's body. That is, on application of the device 300 to a user's body, the proximal surface 304 may be adhered to and be in direct contact with the user contact region. All or portions of the proximal surface 304 may be provided with an adhesive material for maintaining the one or more transducers 306 in direct and close adherence to the user contact region.

The proximal surface 304 may be provided with one or more ultrasound transducers 306 in any suitable arrangement. In the exemplary embodiment shown in FIG. 3B, eighty one transducers are provided in a uniform 9 x 9 rectangular grid arrangement. The transducers are distributed over the backing layer so that ultrasound waves may be transmitted to different portions of the user's body disposed beneath the device 300. In an exemplary embodiment, an ultrasound gel may be provided between the backing layer and the user contact region to improve transmission of the ultrasound waves. The ultrasound gel may be provided around and/or interspersed among the transducers.

In an exemplary embodiment, the backing layer of FIGS. 3A and 3B may take the form of a bandage of any suitable shape. Exemplary shapes may include, but are not limited to, square, rectangular, circular, random, and the like. An exemplary rectangular backing layer may have an exemplary length ranging from about 2 inches to about 24 inches and an exemplary width ranging from about 1 inch to about 12 inches. However, exemplary lengths and widths of the backing layer are not limited to these exemplary ranges.

Exemplary ultrasound transducer devices are not limited to the exemplary number of transducers or the exemplary arrangement shown in FIGS. 3A and 3B.

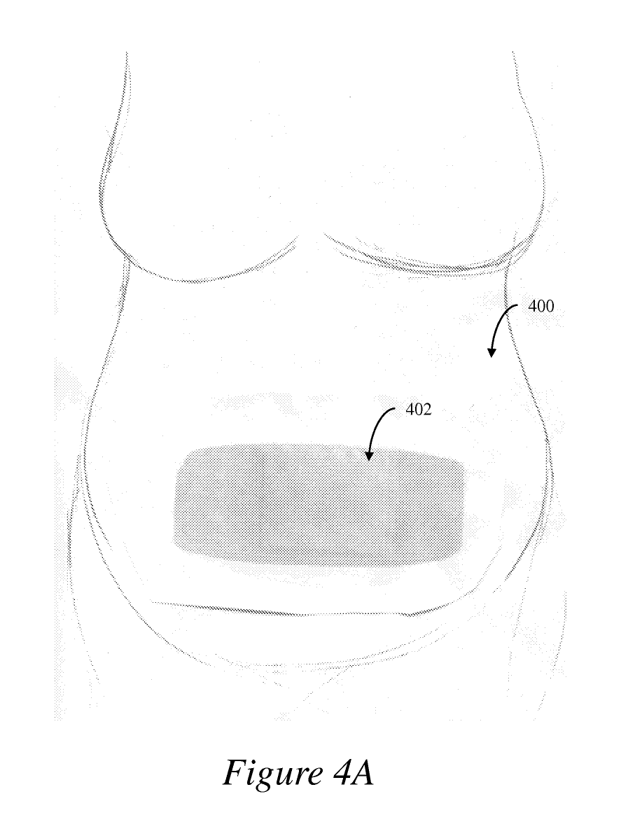

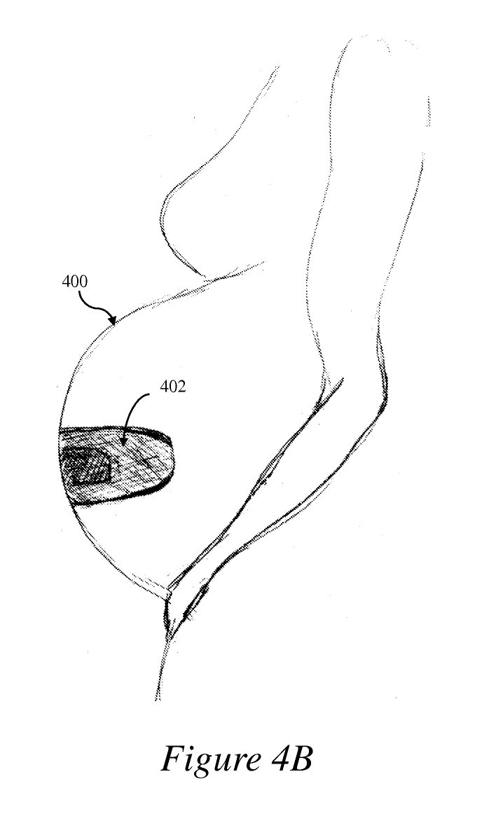

FIG. 4A illustrates a schematic front view of a pregnant woman's abdomen 400 with an exemplary flexible ultrasound transducer device 402 attached to the abdomen for fetal monitoring and/or imaging. FIG. 4B illustrates a schematic side view of the pregnant woman's abdomen 400 with the exemplary flexible ultrasound transducer device 402 of FIG. 4A attached to the abdomen for fetal monitoring.

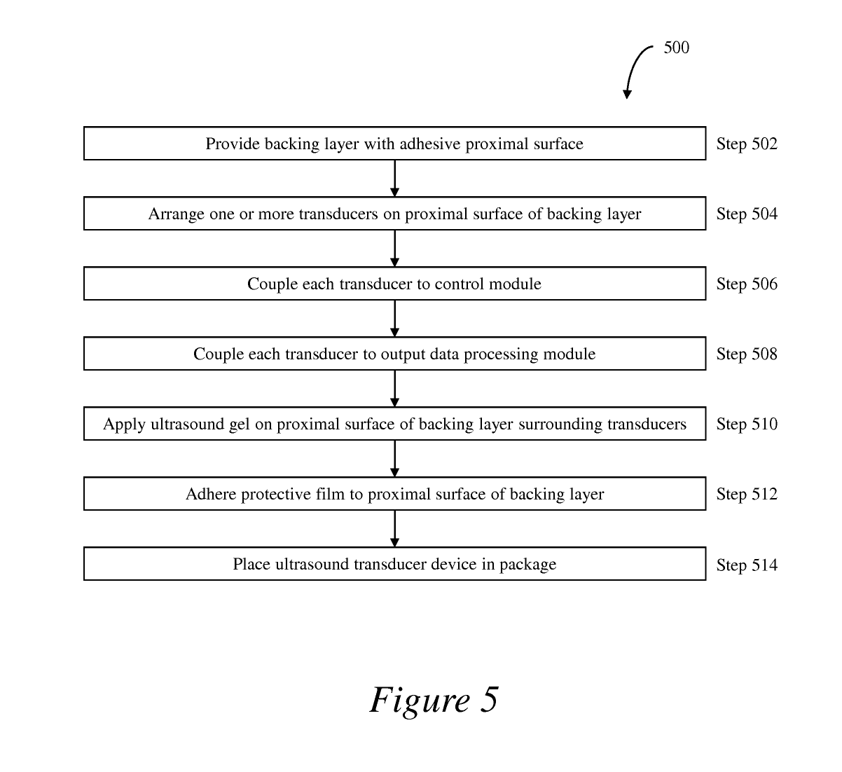

FIG. 5 is a flowchart illustrating an exemplary method 500 for assembling an exemplary flexible ultrasound transducer device.

In step 502, a flat, thin, soft and flexible backing layer may be provided to support one or more ultrasound transducers. In an exemplary embodiment, the backing layer may be a bandage. A proximal surface of the backing layer may be configured for adhering to a user contact region on a user's body. The entirety or one or more portions of the proximal surface of the backing layer may be provided with an adhesive material.

In step 504, one or more ultrasound transducers may be affixed to and arranged on the proximal surface of the backing layer. In an exemplary embodiment, the transducers may be affixed to the proximal surface using the adhesive material on the proximal surface. Alternatively or additionally, one or more transducer attachment mechanisms, e.g., clips, may be provided to affix the transducers to the proximal surface.

In step 506, each transducer may be separately coupled to a control module that controls the function and operation of the transducer. The control module may control, for example, activation of the transducer, deactivation of the transducer, supplying or cutting of power supply to the transducer, frequency settings, duration settings, scan settings, and the like. In an exemplary embodiment, a common control module may be provided to control all of the transducers in the device. In another exemplary embodiment, a plurality of control modules may be provided to separately control all of the transducers or to separately control different subsets of the transducers.

Separate connection of each transducer to the control module may allow selective control over each transducer. For example, one or more transducers, selected by a user or selected automatically, may be deactivated while the remaining transducers are kept activated. Similarly, one or more transducers, selected by a user or selected automatically, may be activated while the remaining transducers are kept deactivated. Power supply to one or more transducers, selected by a user or selected automatically, may be turned on while power supply to the remaining transducers is kept turned off. Similarly, power supply to one or more transducers, selected by a user or selected automatically, may be turned off while power supply to the remaining transducers is kept turned on.

In step 508, each transducer may be separately coupled to an output data processing module. The output data processing module may be programmed to perform one or more data processing activities on the output data received from the transducers.

In an exemplary embodiment, a common output data processing module may be provided to process output data from all of the transducers in the device. In another exemplary embodiment, a plurality of output data processing modules may be provided to separately process output data received from all of the transducers or to separately process output data received from different subsets of the transducers.

Separate connection of each transducer to the output data processing module may allow selective usage of output data from the transducers. For example, output data from one or more transducers, selected by a user or selected automatically, may be used in data processing while output data from the remaining transducers may be excluded. Similarly, output data from one or more transducers, selected by a user or selected automatically, may be excluded in data processing while output data from the remaining transducers may be used.

In step 510, an ultrasound gel may be provided on the proximal surface of the backing layer surrounding the transducers in order to facilitate transmission of ultrasound waves.

In step 512, a sterile protective film may be adhered to the adhesive proximal surface of the backing layer.

In step 514, the ultrasound transducer device may be placed in an outer package, for example, a commercial package.

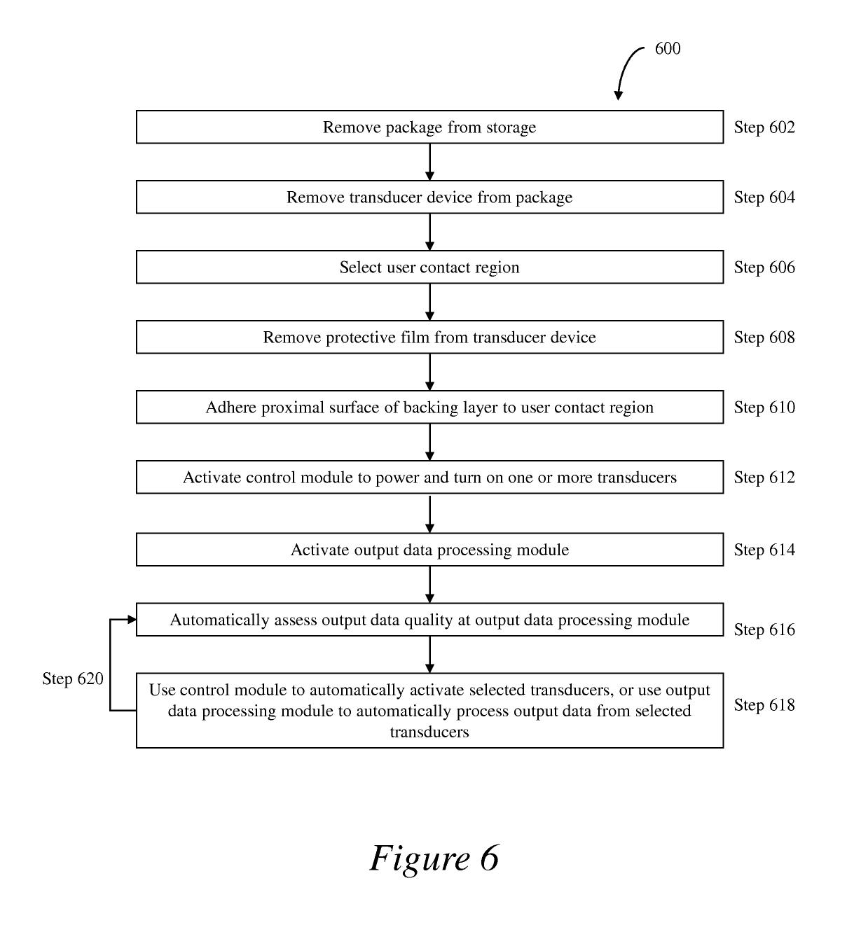

FIG. 6 is a flowchart illustrating an exemplary method 600 for using an exemplary flexible ultrasound transducer device.

In step 602, the packaged flexible ultrasound transducer device may be removed from storage. In step 604, the flexible ultrasound transducer device may be removed from its package and any over-wrap. In step 606, a contact region on the user's body, e.g., the abdomen, may be selected for application of the flexible ultrasound transducer device. In step 608, the protective film covering the adhesive proximal surface of the backing layer may be removed to expose the adhesive surface, the transducers and the gel. In step 610, the proximal surface of the backing layer may be adhered to the contact region on the user's body. This places the transducers and the gel in direct and intimate contact with the user's body.

In step 612, the control module may be activated, and may be used to activate and power on all or a subset of the transducers.

In step 614, the output data processing module coupled to the transducers may be activated. The output data processing module may receive and process output data from the activated transducers in the device. In an exemplary embodiment, the output data processing module may display one or more images generated based on the output data on a display device.

In step 616, the output data processing module may be used to automatically assess the quality, and/or suitability for data processing, of the output data generated by the activated transducers. For example, the output data processing module may automatically determine the quality of an image generated based on output data of a transducer, or the quality of quantitative or qualitative physiological determinations made based on output data of a transducer (e.g., fetal heartbeat, fetal heart rate, fetal breathing rate). The output data processing module may automatically compare the quality assessments for all or a plurality of transducers to determine which transducers are generating output data best suited for monitoring and/or imaging purposes. In an example, only a subset of transducers (e.g., one or more transducers) may be generating the most suitable output data. In another example, all of the transducers in the device may be generating the most suitable output data. The output data processing module may select the best suited transducers and may automatically send an instruction to the control module to indicate the selection of transducers.

Additionally or alternatively, the selection of the transducers may be performed manually by a user. For example, a user may assess one or more images generated based on the output data on a display device, and/or may assess quantitative and qualitative determinations made by the output data processing module. The output data processing module may provide a user interface to display information on which transducers are responsible for the displayed images, determinations and/or measurements. For example, the user interface may show that transducers on the left side of a pregnant woman's abdomen are generating clear output data on fetal heartbeat and/or fetal heart rate, while the transducers on the right side are generated noisy or otherwise unsuitable output data (possibly because the fetus has migrated to the left side of the abdomen). In this case, the user may interact with the user interface to select one or more transducers that are generating output data most suited to the data processing requirements. The output data processing module may automatically send an instruction to the control module to indicate the selection of transducers.

In step 618, in response to the instruction, the control module may deactivate the remaining transducers that were not selected. Additionally or alternatively, in step 618, the output data processing module may exclude output data from the remaining transducers that were not selected. This ensures that only those transducers that are generating output data most suited for output data processing are used to image and/or monitor the user's body.

In step 620, at predefined intervals of time or upon prompt by a user, the quality of the output data of the transducers may be reassessed and the selections of active transducers may be altered, if necessary. For example, the fetus in a pregnant woman's abdomen may shift position over time or the woman herself may shift position. This may result in the output data from one or more transducers that were previously suitable for fetal monitoring no longer being suitable. Step 620 allows the device to automatically and periodically assess the output data quality and selectively use transducers that are generating most suitable output data for fetal monitoring.

### III. Exemplary Arrangements of Ultrasound Transducers

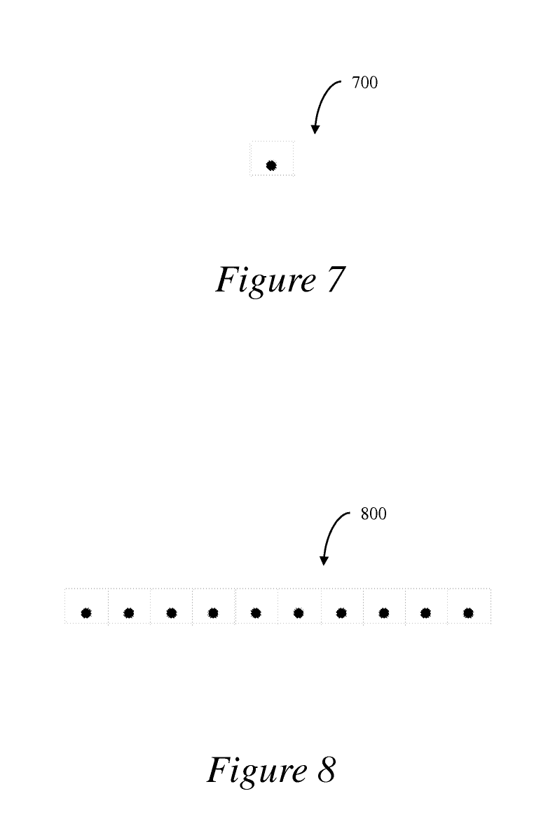

In some exemplary embodiments, an ultrasound transducer array provided in a flexible ultrasound transducer device may include a single ultrasound transducer. FIG. 7 illustrates a schematic of a single exemplary ultrasound transducer 700 provided in a flexible ultrasound transducer device. In an exemplary embodiment, a plurality of single transducers 700 may be separately and selectively placed on a user's body to provide a distributed arrangement of transducers on the body.

In other exemplary embodiments, an ultrasound transducer array may include a plurality of ultrasound transducers ranging, in some embodiments, from two to a million transducers.

In some exemplary embodiments, the transducers may be arranged in any suitable one dimensional, two dimensional or three dimensional configuration including, but not limited to, a strip of transducers arranged linearly, a rectangular grid array, a random array, a circular array, and the like. In one example, the grid array may include two transducers in a 1×1 configuration. In another example, the grid array may include four transducers in a 2×2 configuration. In another example, the grid array may include 900 transducers in a 30×30 configuration. In another example, the grid array may include 1,000,000 transducers in a 1,000×1,000 configuration. In some other examples, the grid array may include any other number and/or configuration of transducers between the 2×1 configuration and the 1,000×1,000 configuration, but are not limited to these exemplary configurations or these exemplary numbers of transducers.

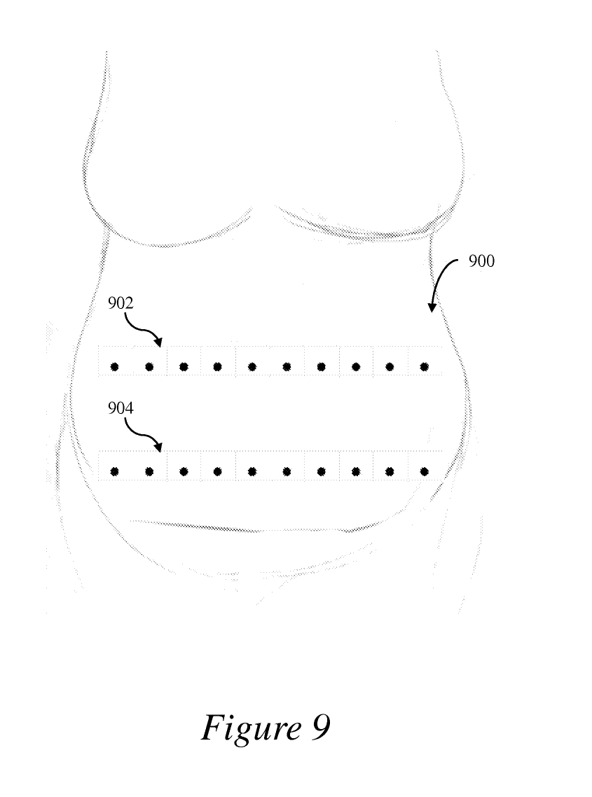

FIG. 8 illustrates a schematic of an exemplary linear strip 800 of a plurality of ultrasound transducers. In this exemplary array, ten ultrasound transducers are arranged in a linear configuration. In an exemplary embodiment, a plurality of strips of transducers may be separately and selectively placed on a user's body to provide a distributed arrangement of transducers on the body. For example, a plurality of the strips may be provided in a consecutive manner to form a rectangular grid array. In another exemplary, the plurality of strips may be spaced from one another, as illustrated in FIG. 9. FIG. 9 is a schematic front view of a pregnant woman's abdomen 900 on which two exemplary linear strips of ultrasound transducers 902 and 904 are placed spaced from each other. Other exemplary strips may be wavy, circular in outline, rectangular in outline, and the like.

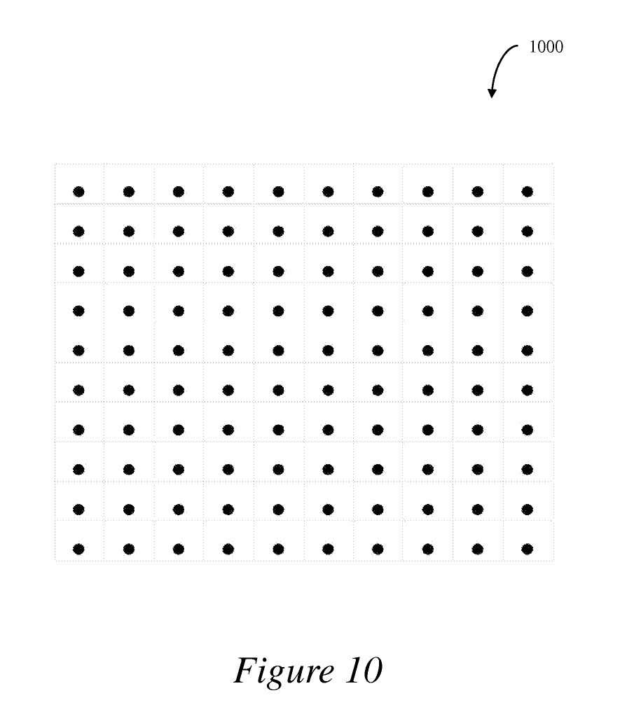

FIG. 10 illustrates a schematic of an exemplary two dimensional grid array 1000 of a plurality of ultrasound transducers. In this exemplary array, one hundred ultrasound transducers are arranged in a 10×10 rectangular grid configuration.

The inter-transducer spacing (i.e., the spacing between consecutive transducers) may range, in some exemplary embodiments, from about 0.1 cm to about 100 cm, but are not limited to this exemplary range. In one example, the inter-transducer spacing may be about 1 cm. The grid configuration may be uniform in some exemplary embodiments so that the inter-transducer spacing is constant across the grid array. In other exemplary embodiments, the inter-transducer spacing may be varied over the grid array depending on the monitoring or imaging needs or the location and/or configuration of the user's body.

In an exemplary embodiment in which a plurality of ultrasound transducers is used, each transducer may include a separate set of electrical connections that couple the transducer to an output data processing module. A selection mechanism may be provided to allow a user or an automated system to connect a selected number of transducers in the array to the output data processing module. The separate connections to the output data processing module may allow use of all of the transducers in the array or a desired subset of the transducers in the output data processing module. The set of transducers that are connected to the output data processing module may be kept the same during an ultrasound monitoring or imaging session, may be changed during the same session, and may be changed between sessions.

In one example, ultrasound waves detected by all of the transducers may be simultaneously used in monitoring an aspect of the user's body or in generating an image of the internal structure of the user's body. In another example, ultrasound waves detected by a selected subset of the transducers may be simultaneously used in monitoring an aspect of the user's body or in generating an image of the internal structure of the user's body. That is, a subset of the transducers in the array may be selected (for example, by a user or automatically), and data from only the selected transducers may be used in the output data processing module. In another example, a subset of the transducers in the array may be selected (for example, by a user or automatically), and data from the selected transducers may be excluded from use by the output data processing module.

### IV. Exemplary Computing Devices

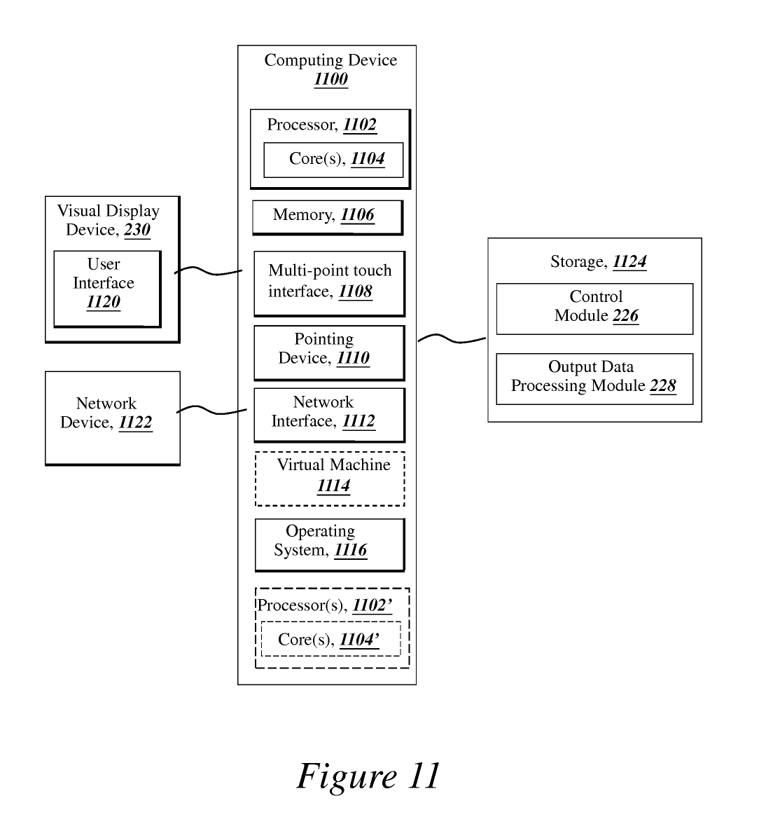

FIG. 11 is a block diagram representing an exemplary computing device 1100 that may be used to perform any of the methods provided by exemplary embodiments. The computing device 1100 may be any computer system, such as an embedded computing system, an embedded processor, a workstation, desktop computer, server, laptop, handheld computer, tablet computer, mobile computing or communication device, or other form of computing or telecommunications device that is capable of communication and that has sufficient processor power and memory capacity to perform the operations described herein. A distributed computational system may be provided comprising a plurality of such computing devices.

The computing device 1100 includes one or more non-transitory computer-readable media having encoded thereon one or more computer-executable instructions or software for implementing exemplary methods. The non-transitory computer-readable media may include, but are not limited to, one or more types of hardware memory, non-transitory tangible media (for example, one or more magnetic storage disks, one or more optical disks, one or more USB flash drives), and the like. For example, memory 1106 included in the computing device 1100 may store computer-readable and computer-executable instructions or software for implementing exemplary embodiments. The computing device 1100 also includes processor 1102 and associated core 1104, and in some embodiments, one or more additional processor(s) 1102′ and associated core(s) 1104′ (for example, in the case of computer systems having multiple processors/cores), for executing computer-readable and computer-executable instructions or software stored in the memory 1106 and other programs for controlling system hardware. Processor 1102 and processor(s) 1102′ may each be a single core processor or multiple core (1104 and 1104′) processor.

Virtualization may be employed in the computing device 1100 so that infrastructure and resources in the computing device may be shared dynamically. A virtual machine 1114 may be provided to handle a process running on multiple processors so that the process appears to be using only one computing resource rather than multiple computing resources. Multiple virtual machines may also be used with one processor.

Memory 1106 may include a computer system memory or random access memory, such as DRAM, SRAM, EDO RAM, and the like. Memory 1106 may include other types of memory as well, or combinations thereof.

A user may interact with the computing device 1100 through a visual display device 230, such as a screen or monitor, that may display one or more user interfaces 1120 that may be provided in accordance with exemplary embodiments. The visual display device 230 may also display other aspects, elements and/or information or data associated with exemplary embodiments. The computing device 1100 may include other I/O devices for receiving input from a user, for example, a keyboard or any suitable multi-point touch interface 1108, a pointing device 1110 (e.g., a mouse, a user's finger interfacing directly with a display device, etc.). The keyboard 1108 and the pointing device 1110 may be coupled to the visual display device 230. The computing device 1100 may include other suitable conventional I/O peripherals.

The computing device 1100 may include one or more storage devices 1124, such as a durable disk storage (which may include any suitable optical or magnetic durable storage device, e.g., RAM, ROM, Flash, USB drive, or other semiconductor-based storage medium), a hard-drive, CD-ROM, or other computer readable media, for storing data and computer-readable instructions and/or software that implement exemplary embodiments as taught herein. For example, the storage device 1124 may provide a control module 226 and an output data processing module 228 (for example, programmed to detect and monitor the heartbeat of a fetus in the user's womb). In an exemplary embodiment, the control module 226 may be provided on a separate storage module or device than the output data processing module 228. The storage device 1124 may be provided on the computing device 1100 or provided separately or remotely from the computing device 1100.

The computing device 1100 may include a network interface 1112 configured to interface via one or more network devices 1122 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1112 may include a built-in network adapter, network interface card, PCMCIA network card, card bus network adapter, wireless network adapter, USB network adapter, modem or any other device suitable for interfacing the computing device 1100 to any type of network capable of communication and performing the operations described herein. The network device 1122 may include one or more suitable devices for receiving and transmitting communications over the network including, but not limited to, one or more receivers, one or more transmitters, one or more transceivers, one or more antennae, and the like.

The computing device 1100 may run any operating system 1116, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 1116 may be run in native mode or emulated mode. In an exemplary embodiment, the operating system 1116 may be run on one or more cloud machine instances.

One of ordinary skill in the art will recognize that the exemplary computing device 1100 may include more or fewer modules than those shown in FIG. 11.

### V. Exemplary Network Environments

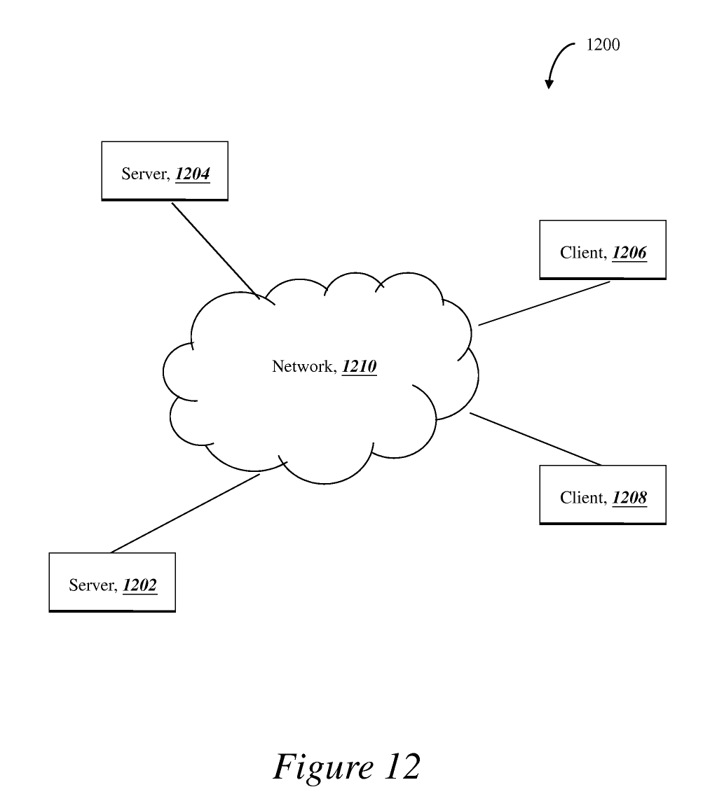

FIG. 12 is a block diagram representing an exemplary network environment 1200 suitable for a distributed implementation of exemplary embodiments. The network environment 1200 may include one or more servers 1202 and 1204 coupled to one or more clients 1206 and 1208 via a communication network 1210. The network interface 1112 and the network device 1122 of the computing device 1100 enable the servers 1202 and 1204 to communicate with the clients 1206 and 1208 via the communication network 1210. The communication network 1210 may include, but is not limited to, the Internet, an intranet, a LAN (Local Area Network), a WAN (Wide Area Network), a MAN (Metropolitan Area Network), a wireless network, an optical network, and the like. The communication facilities provided by the communication network 1210 are capable of supporting distributed implementations of exemplary embodiments.

In an exemplary embodiment, the servers 1202 and 1204 may provide the clients 1206 and 1208 with computer-readable and/or computer-executable components or products or data under a particular condition, such as a license agreement. In an exemplary embodiment, the clients 1206 and 1208 may provide the servers 1202 and 1204 with computer-readable and/or computer-executable components or products or data under a particular condition, such as a license agreement.

In an exemplary embodiment, one or more of the servers 1202 and 1204 and clients 1206 and 1208 may implement a computing system, such as system 1100 or one or more modules thereof as shown in FIG. 11, in order to provide a distributed mechanism for performing the exemplary methods described herein.

In an exemplary distributed implementation, a computing device may be provided at a client 1206 or 1208 remotely from a user undergoing ultrasound monitoring or imaging. For example, the computing device may be provided some distance away from the user in the same room, in a different room, or in a remote location (e.g., a hospital monitoring center). The output data processing module 228 may be provided at a server 1202 or 1204 to provide processed data and/or instructions to the computing device at the client. The processed data may be assessed by a medical practitioner to monitor the physiological condition of the user (e.g., the fetal heartbeat of a fetus in the user's womb) in a remote manner. In an exemplary embodiment, the output data processing module 228 may raise an alarm at the remote computing device at the client upon detection of an alarming condition in the user's body, e.g., an abnormal fetal heartbeat, a high fetal heart rate, lack of fetal movement, and the like.

In another exemplary distributed implementation, the control module 226 may be provided at or may be directly connected to the one or more transducers, and the output data processing module 228 may be provided remotely from the control module 226. For example, the control module 226 may be provided at a client 1206 or 1208, and the output data processing module 228 may be provided at a server 1202 or 1204. During operation of the one or more transducers, the output data processing module at the server may provide one or more instructions to the control module at the client in order to control the operation of the transducers.

One of ordinary skill in the art will recognize that other distributed implementations may be provided using the network environment 1200 of FIG. 12.

### VI. Equivalents

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½nd, and the like, or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than shown.

What is claimed is:

1. A flexible ultrasound transducer device, comprising:
a flexible backing layer having an adhesive proximal surface configured for direct attachment to a user's body and conformable to the user's body; and
a set of one or more ultrasound transducers affixed to the proximal surface of the flexible backing layer and configured so that, upon attachment of the flexible backing layer to the user's body, the one or more ultrasound transducers are maintained in direct and intimate contact with the user's body.

2. The flexible ultrasound transducer device of claim 1, further comprising:
an ultrasound gel provided on the proximal surface of the flexible backing layer and interspersed among the one or more ultrasound transducers so that, upon attachment of the flexible backing layer to the user's body, the ultrasound gel is maintained in direct and intimate contact with the user's body and is prevented from leaking by adherence of edges of the backing layer to the user's body.

3. The flexible ultrasound transducer device of claim 1, further comprising:
an output data processing module coupled to the one or more transducers for receiving output data generated by the one or more transducers, and programmed to monitor one or more physiological aspects of a fetus in the user's body based on the output data.

4. The flexible ultrasound transducer device of claim 3, wherein the one or more physiological aspects of the fetus includes one or more of the following: a heartbeat of the fetus, a structure of the fetus, a location of the fetus, a movement of the fetus, fetal heart rate, breathing of the fetus, lung movement of the fetus, a depth in the user's body of a pocket of amniotic fluid, tone/flexion of the fetus, a reactivity of a heart rate and/or heartbeat of the fetus to movement, and a biophysical profile of the fetus.

5. The flexible ultrasound transducer device of claim 1, wherein the one or more ultrasound transducers are one or more microelectromechanical systems (MEMS) ultrasound transducers.

6. The flexible ultrasound transducer device of claim 1, wherein the one or more ultrasound transducers are one or more piezoelectric ultrasound transducers.

7. The flexible ultrasound transducer device of claim 1, wherein the set of ultrasound transducers includes a plurality of ultrasound transducers configured in a rectangular grid arrangement on the backing layer.

8. The flexible ultrasound transducer device of claim 1, wherein the set of ultrasound transducers includes a plurality of ultrasound transducers configured in a linear strip on the backing layer.

9. The flexible ultrasound transducer device of claim 1, wherein the flexible ultrasound transducer device is reusable between sessions of ultrasound monitoring.

10. The flexible ultrasound transducer device of claim 1, wherein the backing layer is conformable and attachable to any location of the user's body in any desired orientation.

11. The flexible ultrasound transducer device of claim 1, further comprising:
an output data processing module programmed to:
receive a first set of output data from a first transducer within the set of transducers,
receive a second set of output data from a second transducer within the set of transducers,
automatically perform a quality assessment of the first and second sets of output data,
automatically determine that the first set of output data has a higher quality and/or suitability for use in ultrasound data processing than the second set of output data, and
automatically determine that the first transducer is more suitable for the ultrasound data processing than the second transducer.

12. The flexible ultrasound transducer device of claim 11, wherein the output data processing module is further programmed to:
use output data received from the first transducer to perform the ultrasound data processing; and
automatically exclude output data received from the second transducer from the ultrasound data processing.

13. The flexible ultrasound transducer device of claim 11, wherein the output data processing module is further programmed to:
automatically instruct a control module to deactivate the second transducer.

14. The flexible ultrasound transducer device of claim 1, further comprising:
a control module coupled to the one or more transducers and programmed to allow selective deactivation of one or more selected transducers while keeping the remaining transducers activated.

15. A method for performing ultrasound monitoring and/or imaging of a user's body, the method comprising:
providing a flexible ultrasound transducer device, comprising:
a flexible backing layer having an adhesive proximal surface configured for direct attachment to the user's body and conformable to the user's body, and
a set of one or more ultrasound transducers affixed to the proximal surface of the flexible backing layer;
attaching the adhesive proximal surface of the flexible backing layer to the user's body in a conforming manner, so that the one or more ultrasound transducers are maintained in direct and intimate contact with the user's body; and
activating at least one of the one or more transducers in order to perform ultrasound monitoring and/or imaging of the user's body.

16. The method of claim 15, further comprising:
providing an ultrasound gel on the proximal surface of the flexible backing layer and interspersed among the one or more ultrasound transducers so that, upon attachment of the flexible backing layer to the user's body, the ultrasound gel is maintained in direct and intimate contact with the user's body and is prevented from leaking by adherence of edges of the backing layer to the user's body.

17. The method of claim 15, further comprising:
receiving, at an output data processing module coupled to the one or more transducers, output data generated by the one or more transducers; and
monitoring, using the output data processing module, one or more physiological aspects of a fetus in the user's body based on the output data.

18. The method of claim 15, wherein the one or more ultrasound transducers are one or more microelectromechanical systems (MEMS) ultrasound transducers.

19. The method of claim 15, wherein the one or more ultrasound transducers are one or more piezoelectric ultrasound transducers.

20. The method of claim 15, further comprising:
receiving a first set of output data from a first transducer within the set of transducers;
receiving a second set of output data from a second transducer within the set of transducers;
automatically performing a quality assessment of the first and second sets of output data;
automatically determining that the first set of output data has a higher quality and/or suitability for use in ultrasound data processing than the second set of output data, and
automatically determining that the first transducer is more suitable for the ultrasound data processing than the second transducer.

21. The method of claim 20, further comprising:
using output data received from the first transducer to perform the ultrasound data processing; and
automatically excluding output data received from the second transducer from the ultrasound data processing.

22. The method of claim 20, further comprising:
automatically instructing a control module to deactivate the second transducer.

23. The method of claim 15, wherein the ultrasound monitoring and/or imaging of the user's body comprises detecting a heartbeat of a fetus inside the user's womb.
